(19) United States
(12) Patent Application Publication
(10) Pub. No.: US 2016/0058976 A1
(43) Pub. Date: Mar. 3, 2016

(54) INTRODUCER

(71) Applicant: TERUMO KABUSHIKI KAISHA, Tokyo (JP)

(72) Inventors: Ryo OKAMURA, Fujinomiya (JP); Yushin YAZAKI, Fujinomiya (JP)

(73) Assignee: TERUMO KABUSHIKI KAISHA, Tokyo (JP)

(21) Appl. No.: 14/797,442

(22) Filed: Jul. 13, 2015

Related U.S. Application Data
(63) Continuation of application No. PCT/JP2014/051730, filed on Jan. 28, 2014.

(30) Foreign Application Priority Data
Jan. 29, 2013 (JP) 2013-014044

(51) Int. Cl. A61M 25/06; A61M 25/00

(52) U.S. Cl. CPC A61M 25/06; A61M 25/0662; A61M 25/0043; A61M 25/0021; A61M 2025/0059

(57) ABSTRACT

An introducer sheath in which a sheath tube can be easily restored to a state before being kinked even when the sheath tube of the introducer sheath is bent to cause kinking during medical practice. An introducer sheath including: a sheath tube which has a central cavity structure continuing from a proximal portion to a distal portion; and a sheath hub which is connected to the proximal portion of the sheath tube. A return angle of a kinked portion when the sheath tube is bent by 180° is 0° to 15°.

### CROSS-REFERENCES TO RELATED APPLICATIONS

This application is a continuation of International Application No. PCT/JP2014/051730 filed on Jan. 28, 2014, and claims priority to Japanese Application No. 2013-014044 filed on Jan. 29, 2013, the entire content of both of which is incorporated herein by reference.

### TECHNICAL FIELD

The disclosure herein relates to an introducer used for percutaneously introducing a catheter into a blood vessel.

### BACKGROUND DISCUSSION

In recent years, a medical instrument generally called an introducer has been used for introducing a catheter into a blood vessel. The introducer includes a sheath tube having a central cavity structure which is communicated from a proximal portion to a distal portion; and a sheath hub which is provided in the proximal portion of the sheath tube and has the central cavity structure. When introducing a catheter into a blood vessel using the introducer, first, the artery or the vein is punctured with a puncture needle; a guide wire is inserted into the blood vessel through the puncture needle; the puncture needle is removed while leaving the guide wire; the introducer is inserted into the blood vessel such that the introducer in a state in which a dilator is set to be inserted therein covers the periphery of the remaining guide wire; the guide wire and the dilator are removed from the blood vessel while leaving the introducer in the blood vessel; and the introducer is indwelled in the artery or the vein. A diagnostic instrument or a therapeutic instrument is thus put into and taken out of the blood vessel through the indwelled introducer.

When considering the application of such an introducer, it is preferable that the outer diameter of the introducer is small and the structure of the lumen thereof is wide in order to insert the introducer into a lumen in a living body and to insert a diagnostic instrument or a therapeutic instrument into the introducer. Specifically, it is preferable that the sheath tube of the introducer which is inserted into the lumen in the living body has a small outer diameter and a large inner diameter. It is preferable that the wall thickness of the sheath tube is thin. On the other hand, it is preferable that the introducer has a lumen structure with low insertion resistance even when the sheath tube is, for example, bent and kinked, in order to insert medical instruments such as the diagnostic instrument or the therapeutic instrument. Even if the sheath tube is kinked due to pressure from the outside, it is preferable that the kinked portion of the sheath tube is restored to the shape close to the inner diameter of the sheath tube before being kinked, when the pressure from the outside is released. Accordingly, even when the sheath tube is kinked at the time of being inserted into the lumen in a living body, the insertion resistance of the medical instruments into the sheath tube of the introducer is not increased, and therefore, it is possible to easily insert the medical instruments into the lumen of the introducer.

When the wall thickness of the sheath tube is formed thick so as to have the required strength so that the introducer is not kinked, it is possible to prevent the sheath tube from being kinked at the time of being introduced into the skin or the blood vessel, or the like. However, the dimensions of a damage hole formed on the skin or the blood vessel is enlarged, and therefore, puncture resistance during insertion is increased. In addition, in a case where the wall thickness of the sheath tube is thick, it is necessary to limit the size (tube diameter) of the diagnostic instrument or the therapeutic instrument which can be inserted into the introducer. On the other hand, when the wall thickness of the sheath tube is made thin in order to reduce the puncture resistance to a patient, the sheath tube becomes vulnerable. Hence, when the sheath tube is inserted into the skin or the blood vessel, the distal portion of the sheath tube is easily broken, and even if the sheath tube is successfully inserted therein, when the blood vessel of the insertion portion meanders, the sheath tube is bent. The insertion resistance at the time of inserting the diagnostic instrument, therapeutic instrument, or the like into the sheath tube is thereby increased, and as a result, it is difficult to perform insertion or operation of a catheter or the like.

In view of such circumstances, an introducer has been required which has both a small outer diameter and a large inner diameter and which can secure insertion properties of the diagnostic instrument or the therapeutic instrument that is to be inserted into the sheath tube of the introducer during medical practice.

For example, a catheter introducer of which a sheath portion is made of ultra-elastic metal has been proposed as means for solving the problems, as discussed in JP-A-6-225944.

In the case of the structure as disclosed in JP-A-6-225944, since metal has high strength, it is possible to make the wall thickness of an introducer, the sheath portion of which is made of metal, thinner than the wall thickness of an introducer, a sheath tube of which is formed of synthetic resin. Therefore, it is possible to suppress kinking of the introducer. However, the introducer, the sheath portion of which is made of metal, then has little flexibility in the sheath tube. There is thus a concern that the sheath tube may not be bent in accordance with the shape of the blood vessel in a case where the blood vessel meanders, which may cause damage on an inner wall of the blood vessel. For this reason, an introducer has been required which can increase the tube diameter of the inside of the sheath tube while reducing puncture resistance to secure insertion properties of the introducer into a living body; can secure insertion properties of the diagnostic instrument or the therapeutic instrument which is to be inserted into the introducer during medical practice; and has little burden on a patient since there is flexibility in the sheath tube and the inner wall of the blood vessel or the like is hardly damaged.

### SUMMARY

The disclosure here provides an introducer which can increase the tube diameter of the inside of the sheath tube while reducing puncture resistance to secure insertion properties of the introducer into a living body by making the wall thickness thin using synthetic resin in the sheath tube of the introducer; and can secure the flexibility (blood vessel following properties) of the sheath tube. In addition, even when kinking occurs in the sheath tube, the introducer can be naturally restored due to its high resilience so that it is possible to secure insertion properties of the diagnostic instrument or the therapeutic instrument to be inserted into the sheath tube of the introducer during medical practice while enhancing the blood vessel following properties.

An exemplary embodiment of the disclosure is directed to an introducer sheath including: a sheath tube which has a central cavity structure continuing from a proximal portion to a distal portion; and a sheath hub which is connected to the proximal portion of the sheath tube, in which a return angle of a kinked portion when the sheath tube is bent by 180° is 0° to 15°. Here, the expression “return angle of a kinked portion when the sheath tube is bent by 180°” refers to an angle α which is formed of an axis of the sheath tube on a distal side from the kinked portion and an axis of the sheath tube on a proximal side from a kinked portion 30 seconds to 1 minute after the sheath tube is released after one end and the other end of the sheath tube are brought into contact with each other (bent once by 180°) and are completely kinked as shown in FIG. 1.

According to a further aspect of the introducer sheath, the return angle of the kinked portion when the sheath tube is bent by 180° is 2.5° to 12.5°, and preferably the return angle of the kinked portion when the sheath tube is bent by 180° is 2.5° to 7.5°.

In a further aspect of the introducer sheath, a minor axis length/major axis length ratio of a cross section of a return shape of the kinked portion when the sheath tube is bent by 180° is 0.50 to 0.90, preferably, a minor axis length/major axis length ratio of a cross section of a return shape of the kinked portion when the sheath tube is bent by 180° is 0.55 to 0.85, and more preferably, a minor axis length/major axis length ratio of a cross section of a return shape of the kinked portion when the sheath tube is bent by 180° is 0.56 to 0.81.

A further exemplary embodiment of the disclosure is directed to an introducer sheath including: a sheath tube which has a central cavity structure continuing from a proximal portion to a distal portion; and a sheath hub which is connected to the proximal portion of the sheath tube, in which a minor axis length/major axis length ratio of a cross section of a return shape of a kinked portion when the sheath tube is bent by 180° is 0.50 to 0.90. Here, the expression “cross section of a return shape of a kinked portion when the sheath tube is bent by 180°” refers to a shape (FIG. 1(d)) of a cross section of a kinked portion 30 seconds to 1 minute after the sheath tube is released after one end and the other end of the sheath tube are brought into contact with each other (bent once by 180°) and are completely kinked as shown in FIG. 1. In addition, the minor axis of a cross section refers to a length of a shortest straight line when a straight line passing a central point of the sheath tube is drawn inside the kinked cross-section, and the major axis of a cross section refers to a length of the longest straight line when a straight line passing the central point of the sheath tube is drawn inside the kinked cross-section. Note that the central point of the sheath tube is a central axis of a lumen structure of the sheath tube.

Also, the “minor axis length/major axis length” refers to a ratio of the length of the minor axis which is the shortest straight line to the length of the major axis which is the longest straight line, when a straight line passing the center of the portion of the lumen is drawn in the cross section of the kinked portion of the sheath tube of the introducer sheath.

According to a further aspect of the introducer sheath, the minor axis length/major axis length ratio of the cross section of the return shape of the kinked portion when the sheath tube is bent by 180° is 0.55 to 0.85, preferably 0.56 to 0.81.

In a further aspect of the introducer sheath a return angle of the kinked portion when the sheath tube is bent by 180° is 0° to 15°, preferably 2.5° to 12.5°, and more preferably 2.5° to 7.5°.

In another aspect of the introducer sheath, a wall thickness of the sheath tube is 0.050 mm to 0.140 mm, preferably 0.110 mm to 0.140 mm, and more preferably 0.120 mm to 0.140 mm.

Still further, another aspect of the introducer sheath provides an inner diameter of the sheath tube is 1.9 mm to 2.5 mm, preferably 2.0 mm to 2.4 mm.

According to a further aspect of the introducer sheath, an inner diameter/wall thickness ratio of the sheath tube is 13 to 50, preferably 14 to 22, and more preferably 14 to 20.

In another aspect of the introducer sheath, an inner diameter of the sheath tube is 2.3 mm to 2.8 mm, preferably 2.4 mm to 2.7 mm.

Further still, the introducer sheath has an inner diameter/wall thickness ratio of the sheath tube is 16 to 56, preferably 17 to 25, and more preferably 17 to 23.

According to a further aspect of the introducer sheath, a kink generation angle of the sheath tube is 30° to 50°, preferably 30° to 40°. Here, the kink generation angle refers to an angle θ at which kinking occurs in the sheath tube when the sheath tube is pressed by fingers at a position 3 cm away from a proximal side of the sheath tube and the sheath is bent downward as shown in FIG. 6.

A further aspect of the introducer sheath provides that the introducer sheath is formed of ethylene tetrafluoroethylene polymer.

Still further, according to another aspect of the introducer sheath, the introducer sheath has a hemostasis valve incorporated in the sheath hub.

Another exemplary embodiment of the disclosure is directed to an introducer assembly which is formed of the introducer sheath and a dilator.

The following effect can be exhibited when applying the introducer sheath including a sheath tube which has a central cavity structure continuing from a proximal portion to a distal portion; and a sheath hub which is connected to the proximal portion of the sheath tube, in which a return angle of a kinked portion when the sheath tube is bent by 180° is 0° to 15°. That is, even when the sheath tube of the introducer sheath is kinked during treatment, the sheath tube is flexible and has high resilience in which the kinked sheath tube naturally returns to its original condition. Therefore, insertion resistance when inserting a diagnostic instrument or a therapeutic instrument into the sheath tube is lower than that of the introducer in the related art. For this reason, it is possible to continue therapeutic practice without causing a doctor to feel inconvenience even if the kinked introducer sheath is not replaced. As used here, “resilience” refers to properties in which an object returns to its original condition and position when the object is deformed.

In addition, the following effect can be exhibited when applying the introducer sheath including a sheath tube which has a central cavity structure continuing from a proximal portion to a distal portion; and a sheath hub which is connected to the proximal portion of the sheath tube, in which a minor axis length/major axis length ratio of a cross section of a return shape of a kinked portion when the sheath tube is bent by 180° is 0.50 to 0.90. That is, even when the sheath tube of the introducer sheath is kinked during treatment, the sheath tube is flexible and has high resilience in which the kinked sheath tube naturally returns to its original condition. Therefore, insertion resistance when inserting a diagnostic instrument or a therapeutic instrument into the sheath tube is lower than that of the introducer in the related art. For this reason, it is possible to continue therapeutic practice without causing a doctor to feel inconvenience even if the kinked introducer sheath is not replaced.

In addition, the following effect can be exhibited when applying the introducer sheath in which the wall thickness of the sheath tube is 0.050 mm to 0.140 mm. That is, the wall thickness of the sheath tube is thinner than that of the introducer sheath in the related art, and therefore, the outer diameter of the sheath tube is small and the inner diameter of the sheath tube is large. For this reason, in the introducer sheath of the present invention, puncture resistance to the skin or the blood vessel of a patient is reduced and it is possible to insert a diagnostic instrument or a therapeutic instrument, which has a larger tube diameter than that in the related art, into the sheath tube.

### BRIEF DESCRIPTION OF THE DRAWINGS

FIGS. 1(a) to 1(c) are views illustrating a method of testing bending of a sheath tube, and FIG. 1(d) is a cross-sectional view taken along line x-x′ of FIG. 1(c).

FIG. 2 is a plan view of an introducer assembly.

FIG. 3 is a schematic view showing a state in which an introducer sheath is indwelled in the blood vessel.

FIGS. 4(a) to 4(c) are cross-sectional views showing the shapes of three types of introducer sheaths in a normal line direction with respect to an axis direction.

FIGS. 5(a) to 5(h) schematically show a technique of percutaneously inserting an introducer sheath into the blood vessel in order from FIG. 5(a) to FIG. 5(h).

FIG. 6 is a schematic view showing a method of measuring a kink generation angle.

FIG. 7 is a schematic view simply showing an apparatus for a three-point bending test.

FIG. 8 is a schematic view showing a portion of an apparatus of measuring a kink generation curvature radius.

FIGS. 9(a) to 9(c) show a state one minute after introducers having wall thicknesses of 0.12 mm, 0.15 mm, and 0.20 mm, are kinked.

### DETAILED DESCRIPTION

Hereinafter, an exemplary embodiment of the disclosure will be described with reference to the accompanying drawings. Note that the common features are identified by the same reference numerals throughout and the detailed description thereof will not be repeated. In some cases, dimensional ratios in the drawings are exaggerated and are different from the actual ratios for the convenience of description.

An introducer assembly is a device that ensures an access route into a lumen in a living body. Note that, in the description below, the hand operation unit side of the device from which the device is operated will be referred to as a “proximal side”, and the side through which the device is inserted into the lumen in the living body will be referred to as a “distal side”.

A configuration of the introducer assembly to which the introducer sheath according to the exemplary embodiment is applied will be specifically described with reference to FIG. 2.

As shown in FIG. 2, an introducer assembly 1 has an introducer sheath 40 for ensuring an access route into a lumen in a living body, and a dilator 50 that assists percutaneous insertion of the introducer sheath indwelled in the lumen in the living body.

The introducer sheath 40 includes, for example, a sheath tube 41, a sheath hub 42, a hemostasis valve 43, a side port 44, a tube 45, and a three-way stopcock 46. The sheath tube 41 is percutaneously indwelled in the lumen in the living body. Then, a diagnostic instrument or a therapeutic instrument such as a guide wire, a catheter, or the like is inserted into the sheath tube to be introduced into the lumen in the living body. In addition, the sheath hub 42 is attached to the sheath tube 41 on a proximal side, communicates with the sheath tube 41 and the side port 44 therein, and incorporates the hemostasis valve 43. The hemostasis valve 43 is formed of an elastic member that forms a substantially elliptical membrane shape, and is fixed to the sheath hub 42 in a liquid tight manner. In addition, the side port 44 communicates with the sheath tube 41 and the tube 45 and the tube 45 communicates with the side port 44 and the three-way stopcock 46. A liquid such as a physiological salt solution is injected into the introducer sheath through the tube 45 from the three-way stopcock 46.

The dilator 50 includes, for example, a dilator tube 51 and a dilator hub 52. The dilator tube 51 is inserted into the sheath hub 42 and assists percutaneous insertion of the introducer sheath 40 indwelled in the lumen in the living body. In addition, the dilator hub 52 is detachably held at the sheath hub 42. The outer diameter of the dilator tube 51 is substantially the same as or slightly smaller than the inner diameter of the sheath tube 41 since the dilator 50 is to be inserted into the introducer sheath 40.

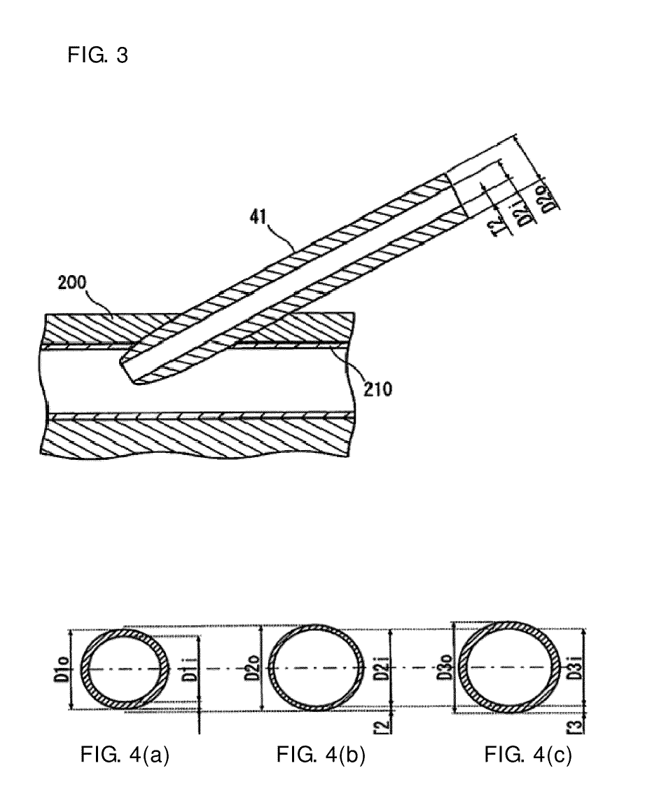

FIG. 3 is a schematic view showing a state in which the introducer sheath is indwelled in the blood vessel. In addition, FIGS. 4(a) to 4(c) are cross-sectional views showing the shapes of three types of introducer sheaths in a normal line direction with respect to an axis direction.

As shown in FIG. 3, it is preferable that the outer diameter of the introducer sheath is made smaller in order to facilitate puncturing in the skin 200 or the blood 210 vessel, or to reduce invasiveness into the vascular endothelium. In addition, it is preferable that the outer diameter of the introducer sheath is smaller in order to quicken the recovery of a puncture site after the operation and to shorten the hemostasis time. Meanwhile, it is also preferable that the inner diameter of the introducer sheath is made larger in order to allow an elongated body with a large diameter be inserted therethrough. For this reason, the introducer sheath preferably has a small outer diameter and a large inner diameter.

The shapes of cross sections of the introducer sheath of the exemplary embodiment and the introducer sheath in the related art are shown in FIGS. 4(a) to 4(c). Here, an outer diameter D2o, an inner diameter D2i, and a wall thickness T2 of the introducer sheath according to the exemplary embodiment of the disclosure are shown in FIG. 4(b). In addition, an outer diameter D1o, an inner diameter D1i, and a wall thickness T1 of the introducer sheath in the related art which has a smaller inner diameter than that of the introducer sheath according to the exemplary embodiment are shown in FIG. 4(a). Similarly, an outer diameter D3o, an inner diameter D3i, and a wall thickness T3 of the introducer sheath in the related art which has a larger outer diameter than that of the introducer sheath according to the present embodiment are shown in FIG. 4(c).

Specifically, the outer diameter D2o of the introducer sheath shown in FIG. 4(b) can be reduced such that the size of the outer diameter D2o thereof is the same as that of the outer diameter D1o of the introducer sheath in the related art shown in FIG. 4(a) of which the size is relatively reduced by 1 Fr (0.33 mm) size. Note that the introducer sheath in the related art shown in FIG. 4(a) corresponds to a 5 Fr size. Here, the introducer sheath with a 5 Fr size refers to an introducer sheath having an inner diameter through which it is possible to insert medical instruments such as a diagnostic instrument or a therapeutic instrument with an outer diameter of a 5 Fr size into a lumen of a sheath tube.

Furthermore, the inner diameter D2i of the introducer sheath shown in FIG. 4(b) can be increased such that the size of the inner diameter D2i thereof is the same as that of the inner diameter D3i of the introducer sheath in the related art shown in FIG. 4(c) of which the size is relatively increased by 1 Fr size. Note that the introducer sheath in the related art shown in FIG. 4(c) corresponds to a 6 Fr size. Here, the introducer sheath with a 6 Fr size refers to an introducer sheath having an inner diameter through which it is possible to insert medical instruments such as a diagnostic instrument or a therapeutic instrument with an outer diameter of a 6 Fr size into a lumen of a sheath tube.

The wall thickness T2 of the introducer sheath shown in FIG. 4(b) can be formed smaller than the wall thickness T1 of the introducer sheath in the related art shown in FIG. 4(a) and the wall thickness T3 of the introducer sheath in the related art shown in FIG. 4(c). In this manner, the introducer sheath shown in FIG. 4(b) can be formed such that the inner diameter D2i thereof is increased by 1 Fr size by reducing the outer diameter D2o thereof by 1 Fr size and by relatively reducing the wall thickness T2 more than that thereof in the related art. The wall thickness of the introducer sheath is formed smaller than the wall thickness T1 and the wall thickness T3.

Hence, the dimension of the outer diameter D2o of the introducer sheath of the exemplary embodiment of the disclosure here is reduced by 1 Fr size by reducing the wall thickness T2 thereof without reducing the dimension of the inner diameter D2i thereof. Accordingly, in the introducer sheath of the exemplary embodiment, it is possible to insert a device, which is the same size as that of a sheath with the outer diameter of a 6 Fr size, into a sheath with the outer diameter D2o of a 5 Fr size. That is, the introducer sheath of the exemplary embodiment has a size of the outer diameter D2o the same as that of the introducer sheath in the related art through which it is possible to insert a medical instrument with an outer diameter of a 5 Fr size, and has a size of the inner diameter the same as that of the introducer sheath in the related art through which it is possible to insert a medical instrument with an outer diameter of a 6 Fr size. For this reason, in the introducer sheath of the exemplary embodiment, it is possible to insert the medical instrument with an outer diameter of a 6 Fr size into the lumen of the sheath tube while the size of the outer diameter of the introducer sheath of the exemplary embodiment is the same as that of the introducer sheath in the related art through which it is possible to insert the medical instrument with an outer diameter of a 5 Fr size.

Such an introducer sheath is denoted as “6 in 5” in consideration of a combination of the 6 Fr size corresponding to the outer diameter of a medical instrument such as a diagnostic instrument or a therapeutic instrument and the 5 Fr size corresponding to the outer diameter of the sheath through which the medical instrument is to be inserted. That is, the aforesaid introducer sheath has the size of the outer diameter the same as that of the introducer sheath in the related art through which it is possible to insert the medical instrument with an outer diameter of a 5 Fr size. However, it is possible to insert the medical instrument with an outer diameter of a 6 Fr size into the lumen of the aforesaid introducer sheath. For this reason, the introducer sheath is denoted as “6 in 5”. Similarly, in the introducer sheath, it is possible to insert a device with an outer diameter of a 7 Fr size into a sheath with an outer diameter D2o of a 6 Fr size. Such an introducer sheath is denoted as “7 in 6” in consideration of a combination of the 7 Fr size corresponding to the outer diameter of a medical instrument and the 6 Fr size corresponding to the outer diameter of the sheath through which the medical instrument is to be inserted. That is, the aforesaid introducer sheath has the size of the outer diameter the same as that of the introducer sheath in the related art through which it is possible to insert the medical instrument with an outer diameter of a 6 Fr size. However, it is possible to insert the medical instrument with an outer diameter of a 7 Fr size into the lumen of the aforesaid introducer sheath. For this reason, the introducer sheath is denoted as “7 in 6”. Similarly, in the introducer sheath, it is possible to insert a device with an outer diameter of a 5 Fr size into a sheath with an outer diameter D2o of a 4 Fr size. Such an introducer sheath is denoted as “5 in 4” in consideration of a combination of the 5 Fr size corresponding to the outer diameter of a medical instrument and the 4 Fr size corresponding to the outer diameter of the sheath through which the medical instrument is to be inserted. That is, the aforesaid introducer sheath has the size of the outer diameter the same as that of the introducer sheath in the related art through which it is possible to insert the medical instrument with an outer diameter of a 4 Fr size. However, it is possible to insert the medical instrument with an outer diameter of a 5 Fr size into the lumen of the aforesaid introducer sheath. For this reason, the introducer sheath is denoted as “5 in 4”.

The introducer sheath corresponding to the aforementioned “6 in 5” has a shape in which the inner diameter thereof is 1.9 mm to 2.5 mm, and the wall thickness thereof is 0.050 mm to 0.140 mm. The inner diameter of the sheath is preferably 2.0 mm to 2.4 mm and the wall thickness of the sheath is preferably 0.110 mm to 0.140 mm. The inner diameter of the sheath is more preferably 2.0 mm to 2.4 mm and the wall thickness of the sheath is more preferably 0.120 mm to 0.140 mm. The ratio of the inner diameter to the wall thickness of the introducer sheath, that is, the inner diameter/wall thickness ratio thereof is 13 to 50, preferably 14 to 22, and more preferably 14 to 20.

The introducer sheath corresponding to the aforementioned “7 in 6” has a shape in which the inner diameter thereof is 2.3 mm to 2.8 mm and the wall thickness thereof is 0.050 mm to 0.140 mm. The inner diameter of the sheath is preferably 2.4 mm to 2.7 mm and the wall thickness of the sheath is preferably 0.110 mm to 0.140 mm. The inner diameter of the sheath is more preferably 2.4 mm to 2.7 mm and the wall thickness of the sheath is more preferably 0.120 mm to 0.140 mm. The ratio of the inner diameter to the wall thickness of the introducer sheath, that is, the inner diameter/wall thickness ratio thereof is 16 to 56, preferably 17 to 25, and more preferably 17 to 23.

The introducer sheath corresponding to the aforementioned “5 in 4” has a shape in which the inner diameter thereof is 1.5 mm to 2.2 mm and the wall thickness thereof is 0.050 mm to 0.140 mm. The inner diameter of the sheath is preferably 1.6 mm to 2.1 mm and the wall thickness of the sheath is preferably 0.110 mm to 0.140 mm. The inner diameter of the sheath is more preferably 1.6 mm to 2.1 mm and the wall thickness of the sheath is more preferably 0.120 mm to 0.140 mm. The ratio of the inner diameter to the wall thickness of the introducer sheath, that is, the inner diameter/wall thickness ratio thereof is 11 to 44, preferably 12 to 20, and more preferably 12 to 18.

The material constituting the sheath tube may be, for example, a polymer material such as polyolefin (for example, polyethylene, polypropylene, polybutene, an ethylene-propylene copolymer, an ethylene-vinyl acetate copolymer, an ionomer, and a mixture of at least two thereof), a polyolefin elastomer, a cross-linked body of polyolefin, polyvinyl chloride, polyamide, a polyamide elastomer, polyester, a polyester elastomer, polyurethane, a polyurethane elastomer, fluorine resin, polycarbonate, polystyrene, polyacetal, polyimide, and polyetherimide, and a mixture thereof. Preferably, an ethylene tetrafluoroethylene copolymer (ETFE) is most suitable in consideration of the effect of resilience in which the kinked sheath tube returns to its original condition.

In the introducer sheath according to the disclosure here, a return angle of a kinked portion when the sheath tube is released after being bent once by 180° is 0° to 15°, preferably 2.5° to 12.5°, and more preferably 2.5° to 7.5°. In addition, in the introducer sheath, a minor axis length/major axis length ratio of a cross section of a return shape of the kinked portion when the sheath tube is bent by 180° is 0.50 to 0.90, preferably 0.55 to 0.85, and more preferably 0.56 to 0.81. When such a configuration is used, the sheath tube is more likely to be kinked since the wall thickness of the introducer sheath is thinner than that thereof in the related art. However, even if the sheath tube is kinked, the sheath tube has high resilience, and insertion resistance when inserting a medical instrument into the introducer can be prevented from being increased, and therefore, it is possible to comfortably proceed with medical practice.

In addition, in the introducer sheath according to the disclosure here, the tensile strength is greater than or equal to 4.0, and the yield strength is greater than or equal to 1.53 kgf (in ISO11070, greater than or equal to 15 N (≅greater than or equal to 1.53 kgf) of Force at Break). For this reason, the possibility that the sheath tube is broken and damaged is extremely low even if the wall thickness of the introducer sheath is made thin.

Next, a technique of percutaneously inserting the introducer sheath according to the exemplary embodiment into the blood vessel will be described.

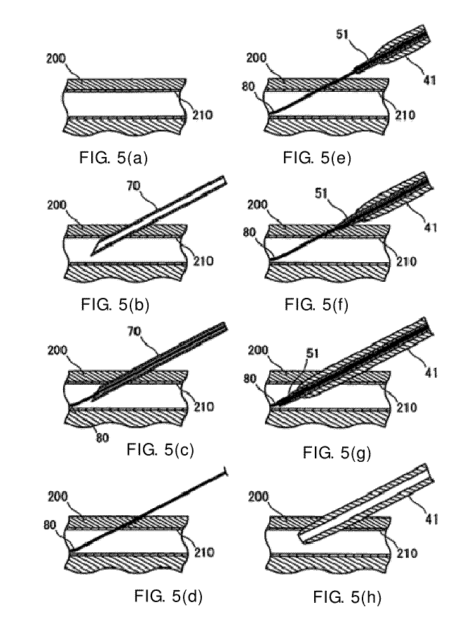

A sheath tube 41 of the introducer sheath is inserted into a blood vessel 210, which is positioned below a skin 200 shown in FIG. 5(a), beyond the skin. Specifically, first, the skin 200 is punctured with a puncture needle 70 toward the blood vessel 210 as shown in FIG. 5(b). Next, a guide wire 80 is inserted in the blood vessel through a lumen of the puncture needle 70 as shown in FIG. 5(c). Next, the puncture needle 70 is removed from inside of the blood vessel while the guide wire 80 is indwelled in the blood vessel as shown in FIG. 5(d). Thereafter, the dilator tube 51 which is mounted with the sheath tube 41 is inserted into the blood vessel through the skin 200 along the guide wire 80 as continuously shown in FIGS. 5(e) to 5(g). Next, the guide wire 80 and the dilator tube 51 are removed from inside of the blood vessel while the sheath tube is indwelled in the blood vessel as shown in FIG. 5(h). Then, a therapeutic instrument or a diagnostic instrument (catheter) which is not shown in the drawing is inserted into the sheath tube 41.

Next, the exemplary embodiment of the disclosure will be more specifically described using examples, but the disclosure herein is not limited to these examples.

### Example 1

### Manufacture of Introducer Sheath—0.12 mm Wall Thickness

First, fluorine resin (ethylene tetrafluoroethylene copolymer) was extruded into a tubular shape at the outer periphery of a columnar core material through hollow extrusion molding to manufacture a sheath tube with 2.22 mm inner diameter×2.46 mm outer diameter (wall thickness: 0.12 mm). Next, an acrylamido-glycidyl methacrylate copolymer (lubricant material) was covered on the outer periphery of the sheath tube which was then subjected to tip processing (taper processing). The processed sheath tube was then mounted on a separately manufactured sheath hub. Accordingly, an introducer sheath was obtained. Note that the introducer sheath is the above-described “6 in 5” type.

### Comparative Example 1

### Manufacture of Introducer Sheath—0.15 mm Wall Thickness

Fluorine resin (ethylene tetrafluoroethylene copolymer) was extruded into a tubular shape at the outer periphery of a columnar core material through hollow extrusion molding to manufacture a sheath tube with 2.22 mm inner diameter×2.52 mm outer diameter (wall thickness: 0.15 mm). An introducer sheath was obtained in the same manner as in Example 1 except for the size of the sheath tube.

### Comparative Example 2

### Manufacture of Introducer Sheath—0.20 mm Wall Thickness

Fluorine resin (ethylene tetrafluoroethylene copolymer) was extruded into a tubular shape at the outer periphery of a columnar core material through hollow extrusion molding to manufacture a sheath tube with 2.22 mm inner diameter×2.62 mm outer diameter (wall thickness: 0.20 mm). An introducer sheath was obtained in the same manner as in Example 1 except for the size of the sheath tube.

### Example 2

### Measurement of Dimension of Sheath Tube and Measurement of Kink Generation Angle

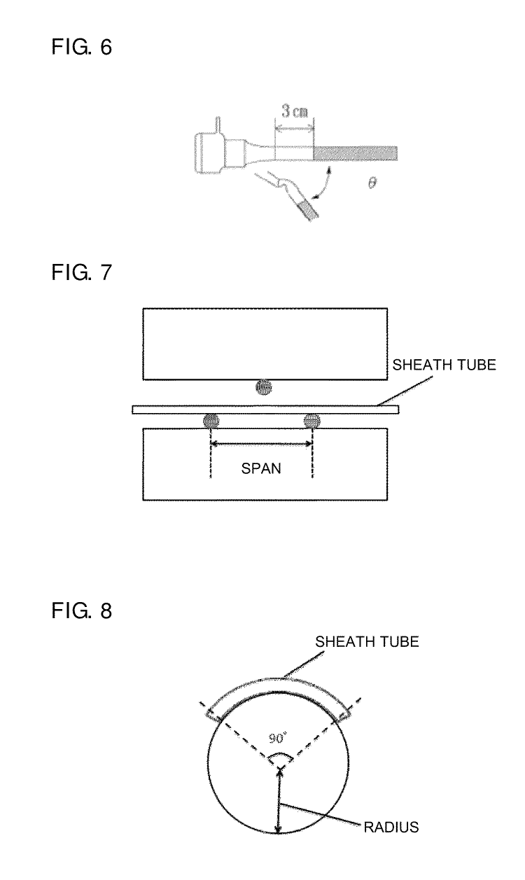

Actual measurement values of the inner diameter, the outer diameter, and the wall thickness in the sheath tube of the introducer sheath manufactured in Example 1 were measured using a projector. In addition, the kink generation angle of the introducer sheath manufactured in Example 1 was measured 5 times. Here, the kink generation angle refers to an angle θ at which kinking occurs in the sheath tube when the sheath tube is pressed by fingers at a position 3 cm away from a proximal side of the sheath tube and the sheath is bent downward as shown in FIG. 6.

TABLE 1

| | Inner diameter | Outer diameter | Wall thickness |
|---|---|---|---|
| | (mm) | (mm) | (mm) |
| (1) | 2.190 | 2.465 | 0.140 |
| (2) | 2.190 | 2.460 | 0.135 |
| (3) | 2.190 | 2.445 | 0.125 |
| (4) | 2.180 | 2.445 | 0.135 |
| (5) | 2.190 | 2.450 | 0.130 |
| (6) | 2.185 | 2.450 | 0.130 |
| (7) | 2.195 | 2.450 | 0.125 |
| (8) | 2.190 | 2.435 | 0.120 |
| (9) | 2.185 | 2.450 | 0.130 |
| (10) | 2.190 | 2.445 | 0.130 |
| Average value | 2.189 | 2.450 | 0.130 |
| Standard deviation | 0.004 | 0.008 | 0.006 |

TABLE 2

| | Kink generation angle (°) |
|---|---|
| (1) | 30 |
| (2) | 35 |
| (3) | 30 |
| (4) | 30 |
| (5) | 35 |
| Average value | 32 |
| Standard deviation | 2.7 |

In regard to the introducer sheath according to the disclosure here, the actual measurement values of the dimension of the sheath tube are shown in Table 1 and the kink generation angles are shown in Table 2. The actual measurement value of the wall thickness of the introducer sheath manufactured in Example 1 is between 0.120 mm and 0.140 mm. In addition, the kink generation angle of the sheath tube is 30° to 35° and it was confirmed that the sheath tube has flexibility while having kink resistance with respect to pushing force from the outside.

Next, the introducer sheaths manufactured in Example 1 and Comparative Examples 1 and 2 were tested as shown in the following Examples 3 to 8.

### Example 3

### Tensile Strength and Yield Strength of Sheath Tube

The tensile strengths and the yield strengths of the introducer sheaths manufactured in Example 1 and Comparative Examples 1 and 2 were measured under the following measurement conditions using Autograph (AG-X plus, Shimidazu Corporation, the same applies hereinafter).

### Measurement Conditions

Tensile speed: 200 mm/min
Inter-fulcrum distance (Chuck distance): 10 mm

TABLE 3

| | | |
|---|---|---|
| t = 0.12 | t = 0.15 | t = 0.20 |

| Yield | Tensile | Yield | Tensile | Yield | Tensile |
|---|---|---|---|---|---|
| strength | strength | strength | strength | strength | strength |
| (kgf) | (kgf) | (kgf) | (kgf) | (kgf) | (kgf) |
| ◯ | ◯ | ◯ | ◯ | ◯ | ◯ |

t: Wall thickness
◯: Yield strength or tensile strength is greater than or equal to ISO standard
X: Yield strength or tensile strength is less than ISO standard

The tensile strengths and the yield strengths of the sheath tubes of the introducer sheaths manufactured in Example 1 and Comparative Examples 1 and 2 are as shown in Table 3. The yield strengths of the introducer sheaths with respective wall thicknesses exceeded ISO 11070 (greater than or equal to 15 N (≅greater than or equal to 1.53 kgf) of Force at Break) which is an ISO standard. For this reason, even if the wall thickness of the introducer sheath is made thin, there is low risk that the sheath tube may be broken and damaged during medical practice. Hence, even if the wall thickness of the introducer sheath according to the disclosure is made thin, there is low risk that the sheath tube may be broken and damaged due to standard therapeutic practice. Here, the tensile strength refers to strength of an endurance limit without fracturing when an object is pulled in a certain direction. The yield strength refers to strength when plastic deformation starts when an object is pulled in a certain direction.

### Example 4

### Three-Point Bending Test

The kinked-point strengths (gf) of the introducer sheaths manufactured in Example 1 and Comparative Examples 1 and 2 were measured 3 times under the following measurement conditions using Autograph by applying a load to the center between the fulcrums on the sheath tube. FIG. 7 is a schematic view illustrating an apparatus used in a three-point bending test.

### Measurement Conditions

Pushing speed: 5 mm/min
Inter-fulcrum distance (span): 25.4 mm

TABLE 4

| | | t = 0.12 | t = 0.15 | t = 0.20 |
|---|---|---|---|---|
| Kink strength (gf) | (1) | 90.0 | 153.9 | 241.7 |
| | (2) | 89.7 | 155.2 | 242.3 |
| | (3) | 84.3 | 150.3 | 243.1 |

| | | | |
|---|---|---|---|
| Average value (gf) | 88.0 | 153.1 | 242.4 |

t: Wall thickness

### Example 5

### Measurement of Kink Generation Curvature Radius

In regard to the introducer sheaths manufactured in Example 1 and Comparative Examples 1 and 2, the maximum ellipticity radius at which kinking occurs when the sheath tube is bent by 90° of a curvature radius of a circular member was measured using the circular member with a radius of 24 mm to 25 mm. Specifically, the sheath tube was placed along the circular member using a device as shown in FIG. 8, and the existence of the occurrence of kinking of the sheath tube was checked in a case where the sheath tube placed along the circular member was bent by 90° with respect to the center of the circular member.

TABLE 5

| | t = 0.12 | t = 0.15 | t = 0.20 |
|---|---|---|---|
| Kink generation | less than or | less than or equal | less than or equal |
| curvature radius | equal to 25 mm | to 25 mm | to 25 mm |

t: Wall thickness

In regard to the introducer sheaths manufactured in Example 1 and Comparative Examples 1 and 2, the kinked-point strengths of the sheath tubes are shown in Table 4, and the kink generation curvature radiuses of the sheath tubes are shown in Table 5. As shown in Table 4, the kink resistance was reduced as the wall thickness of the sheath tube became thinner. However, in the kink generation curvature radius, the sheath tube was not kinked even at a kink generation curvature radius of 25 mm. For this reason, in the introducer sheath according to the disclosure here, the kinked-point strength is low since the wall thickness of the introducer sheath is thin, but the sheath has sufficient flexibility. For example, there is a technique called Trans Radial Intervention (TRI). In this technique, an introducer sheath is indwelled in a radial artery, and a medical instrument is introduced into the brachial artery or the aorta from the radial artery. In the case of this technique, assuming that a sheath tube is inserted into a lumen in a living body at a puncture angle of 30° which is standard in TRI, and that a sheath is fixed and bent by the skin and a blood vessel wall, the curvature radius thereof can be calculated as about 80 mm. For this reason, there is a low risk that the kinking may occur at least in the TRI technique. Here, the kinked-point strength indicates pushing force (gf) required when the sheath tube of the introducer sheath is kinked.

### Example 6

### Measurement of Return Angle after being Kinked and Measurement of Inner Diameter after being Kinked

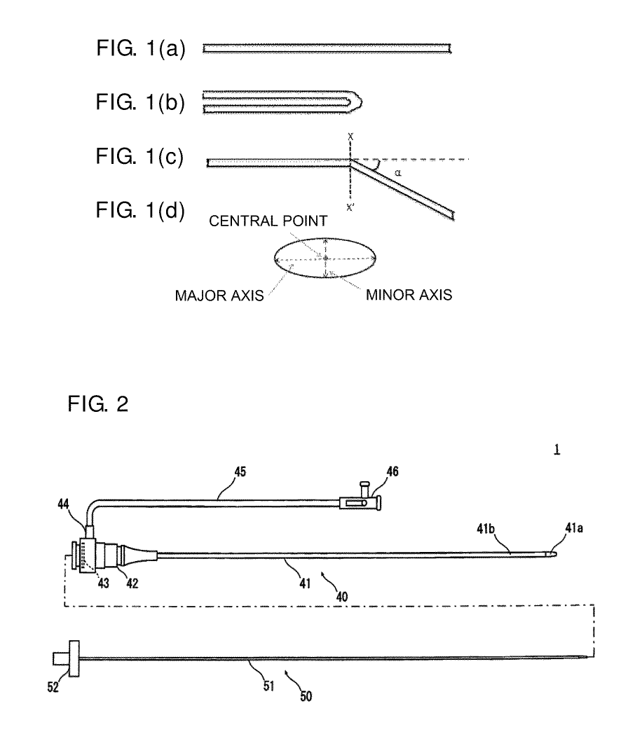

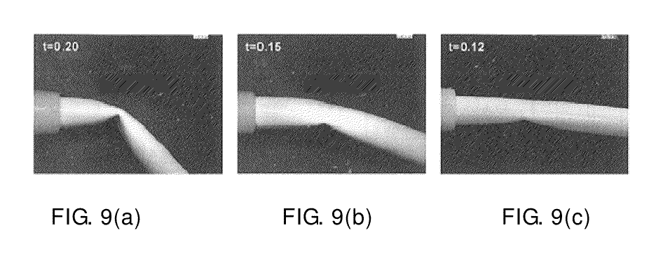

In regard to the introducer sheaths manufactured in Example 1 and Comparative Examples 1 and 2, the return angle α of the kinked portion one minute after the sheath tube was bent once by 180° as shown in FIGS. 1(a) to 1(c) was measured 6 times by units of 5° to check which range the return angle α belongs to (FIGS. 9(a) to 9(c)). Specifically, the “return angle of the kinked portion” refers to an angle α which is formed of an axis of the sheath tube on a distal side from the kinked portion and an axis of the sheath tube on a proximal side from a kinked portion 30 seconds to 1 minute after the sheath tube is released after one end and the other end of the sheath tube are brought into contact with each other (bent once by 180°) and are completely kinked as shown in FIG. 1(b). In addition, a silicon mold of the kinked portion at each of return angles of 5°, 20°, and 45° was measured 6 times using a laser outer diameter measurement device, and each length of the minor axis and the major axis of substantially elliptical shapes of the kinked cross-sections was measured (the diameter of the circular cross section before being kinked was 2.46 mm). Here, the minor axis of the kinked cross-section refers to a length of the shortest straight line when a straight line passing a central point of the sheath tube is drawn inside the kinked cross-section. In addition, the major axis of the kinked cross section refers to a length of the longest straight line when a straight line passing the central point of the sheath tube is drawn inside the kinked cross-section. Note that the central point of the sheath tube is a central axis of a lumen structure of the sheath tube. Also, note that the “minor axis length/major axis length” refers to a ratio of the length of the minor axis which is the shortest straight line to the length of the major axis which is the longest straight line, when a straight line passing the center of the portion of the lumen is drawn in the cross section of the kinked portion of the sheath tube of the introducer sheath.

TABLE 6

| | | t = 0.12 | t = 0.15 | t = 0.20 |
|---|---|---|---|---|
| Return angle (°) | (1) | 2.5-7.5 | 17.5-22.5 | 42.5-47.5 |
| | (2) | 2.5-7.5 | 22.5-27.5 | 42.5-47.5 |
| | (3) | 7.5-12.5 | 17.5-22.5 | 42.5-47.5 |
| | (4) | 2.5-7.5 | 22.5-27.5 | 37.5-42.5 |
| | (5) | 2.5-7.5 | 17.5-22.5 | 42.5-47.5 |
| | (6) | 2.5-7.5 | 17.5-22.5 | 47.5-52.5 |

t: Wall thickness

TABLE 7

| | 5° | | | 20° | | | 45° | | |
|---|---|---|---|---|---|---|---|---|---|
| | | | Minor | | | Minor | | | Minor |
| | | | axis | | | axis | | | axis |
| | | | length/ | | | length/ | | | length/ |
| | Minor | Major | Major | Minor | Major | Major | Minor | Major | Major |
| | axis | axis | axis | axis | axis | axis | axis | axis | axis |
| | (mm) | (mm) | length | (mm) | (mm) | length | (mm) | (mm) | length |
| (1) | 1.6762 | 2.6183 | 0.64 | 0.987 | 3.186 | 0.31 | 0.713 | 3.239 | 0.22 |
| (2) | 1.9623 | 2.4349 | 0.81 | 1.101 | 3.014 | 0.37 | 0.786 | 3.218 | 0.24 |
| (3) | 1.5357 | 2.7125 | 0.57 | 1.131 | 3.010 | 0.38 | 0.864 | 3.399 | 0.25 |
| (4) | 1.5680 | 2.6685 | 0.59 | 0.969 | 3.194 | 0.30 | 0.975 | 3.187 | 0.31 |
| (5) | 1.6903 | 2.5888 | 0.65 | 1.024 | 3.036 | 0.34 | 1.044 | 3.156 | 0.33 |
| (6) | 1.5268 | 2.7070 | 0.56 | 1.038 | 3.182 | 0.33 | 0.807 | 3.155 | 0.26 |
| Average | 1.66 | 2.62 | 0.63 | 1.04 | 3.10 | 0.34 | 0.86 | 3.23 | 0.27 |
| value | | | | | | | | | |

The return angles of the kinked portion of the sheath tubes of the introducer sheaths manufactured in Example 1 and Comparative Examples 1 and 2 are shown in Table 6. For example, the return angle “2.5 to 7.5” in Table 6 indicates that the return angle is in a range of greater than or equal to 2.5° and less than 7.5°. In addition, the minor axis length, major axis length, and minor axis length/major axis length of the cross section of the kinked portion when the return angles of the sheath tubes are 5°, 20°, and 45° are shown in Table 7. From these results, the return angle was reduced as the wall thickness of the sheath tube became thinner; when the wall thickness of the sheath tube was 0.12 mm, the return angle mostly fell within a range of greater than or equal to 2.5° and less than 7.5°; when the wall thickness of the sheath tube was 0.15 mm, the return angle mostly fell within a range of greater than or equal to 17.5° and less than 22.5°; and when the wall thickness of the sheath tube was 0.20 mm, the return angle mostly fell within a range of greater than or equal to 42.5° and less than 47.5°. In addition, the minor axis length/major axis length ratio which is proportional to the size of the inner diameter is increased as the return angle of the sheath tube is reduced. Therefore, it was found that the sheath tube with a wall thickness of 0.12 mm had high resilience, in which the kinked sheath tube naturally returns to its original shape, compared to other tubes. That is, the sheath tube (0.120 mm to 0.140 mm wall thickness) of Example 1 made of ethylene tetrafluoroethylene copolymer which a fluorine resin had high resilience, in which the kinked portion of the sheath tube naturally returns to an original shape of the lumen before being kinked, compared to the sheath tubes of Comparative Examples 1 and 2. For this reason, when the introducer sheath is inserted into a lumen in a living body, even if the sheath tube is kinked due to the meandering blood vessel, the shape of the lumen of the sheath tube tends to return to an original shape of the lumen before being kinked. Accordingly, in the introducer sheath of the disclosure here, it is possible to easily insert a medical instrument into the sheath tube even when the sheath tube is kinked in the lumen in the living body.

In the following Examples 6 to 8, the performance of the introducer sheath (wall thickness: 0.12) according to the exemplary embodiment of the disclosure was confirmed by comparing insertion resistance of the introducer sheath according to the exemplary embodiment with insertion resistances of conventional products (wall thickness: 0.15 mm and 0.20 mm), when inserting a medical instrument into a kinked sheath tube.

### Example 7

### Measurement of Insertion Resistance when Inserting Dilator

Resistance values (gf) of the introducer sheaths manufactured in Example 1 and Comparative Examples 1 and 2 when a distal end of a dilator passed through a kinked portion were measured three times by inserting the dilator into a kinked sheath tube after releasing the sheath tube which had been bent and kinked once by 180°.

TABLE 8

| | t = 0.12 | t = 0.15 | t = 0.20 | Valve body |
|---|---|---|---|---|
| (1) | 59.5 | 85.8 | 198.9 | 64.2 |
| (2) | 36.4 | 91.5 | 133.4 | 58.0 |
| (3) | 39.2 | 89.3 | 176.1 | 64.5 |
| Average value | 45.0 | 88.9 | 169.5 | 62.2 |

t: Wall thickness

The resistance values of the introducer sheaths manufactured in Example 1 and Comparative Examples 1 and 2 when the dilator passed through the kinked portion after the sheath tube was kinked were measured, and the results are shown in Table 8. As a result, it was found that, when the wall thickness was 0.12 mm, even if the sheath tube was kinked, the insertion resistance of the kinked portion was lower than the insertion resistance value when the dilator passed through the valve body. For this reason, a force on a hand of a doctor or a force on the distal portion of a dilator tube is lower than the force when inserting the dilator into the valve body. Therefore, even if a doctor or the like uses the introducer sheath in a state of being kinked, there is no case where the doctor feels more resistance than necessary in the hand.

### Example 8

### Measurement of Insertion Resistance when Inserting Guiding Catheter

The resistance values (gf) of the introducer sheaths manufactured in Example 1 and Comparative Examples 1 and 2 when a distal end of a guiding catheter (Heartrail II, Terumo Corporation) passed through a kinked portion were measured three times by inserting the guiding catheter into a kinked sheath tube after releasing the sheath tube which had been bent and kinked once by 180°.

TABLE 9

| | t = 0.12 | t = 0.15 | t = 0.20 |
|---|---|---|---|
| (1) | 115.5 | 208.9 | 361.1 |
| (2) | 129.3 | 222.6 | 339.8 |
| (3) | 142.6 | 210.2 | 353.5 |
| Average value | 129.1 | 213.9 | 351.5 |

t: Wall thickness

The resistance values of the introducer sheaths manufactured in Example 1 and Comparative Examples 1 and 2 when the guiding catheter passed the kinked portion after the sheath tube is kinked were measured, and the results thereof are shown in Table 9. As a result, it was found that the insertion resistance when the guiding catheter passed through the kinked portion was reduced as the wall thickness of the sheath tube became thinner. For this reason, even when the sheath tube of the introducer sheath of the disclosure here is kinked, the sheath tube has high resilience in which the kinked sheath tube returns to the condition before being bent. Therefore, insertion of the medical instrument such as the guiding catheter into the kinked sheath tube does not apply a large force onto the hand of a doctor and does not hinder the medical practice.

According to the above-described exemplary embodiment of an introducer sheath according to the disclosure, the following effects can be exhibited.

When inserting an introducer sheath into a lumen in a living body of a patient, the introducer sheath of the exemplary embodiment exhibits various effects in terms of the shape of the introducer sheath. The effects thereof will be specifically described.

When inserting an introducer sheath into a lumen in a living body of a patient, even if a sheath tube of the introducer sheath is kinked, the inner diameter of the kinked portion of the sheath tube naturally returns to the condition close to the inner diameter thereof before being kinked. For this reason, an increase in the insertion resistance in the sheath tube when inserting a dilator into the sheath tube is suppressed. In addition, when inserting an introducer assembly which is obtained by inserting the dilator into the introducer sheath, the shape of the inner diameter of the kinked portion of the introducer sheath of the exemplary embodiment easily recovers to the shape of the inner diameter before being kinked, compared to the introducer sheath in the related art. Therefore, puncture resistance when inserting the introducer sheath into the lumen in the living body is also suppressed.

Even when the sheath tube of the introducer sheath is kinked after the introducer sheath is inserted into a lumen in a living body of a patient, the inner diameter of the kinked portion of the sheath tube naturally recovers to the inner diameter before being kinked. Therefore, an increase in the insertion resistance when a diagnostic instrument, a therapeutic instrument or the like is inserted into the sheath tube is suppressed. For this reason, even when the introducer sheath is kinked during medical practice, the insertion resistance to the inside of the sheath tube of the introducer sheath is not increased, and the medical practice is easily continued without exchanging the introducer sheath, and therefore, the stress on a patient can be reduced.

Furthermore, the following effect is exhibited since the wall thickness of the sheath tube of the introducer sheath having the aforesaid effects is thin.

In the introducer sheath of the exemplary embodiment, the outer diameter thereof is reduced by 1 Fr size, and therefore, an insertion mark remaining when percutaneously inserting the introducer sheath into the blood vessel or the like is relatively small. Accordingly, it is possible to shorten the time required for hemostasis. Specifically, the outer diameter of the sheath tube of the introducer sheath of the exemplary embodiment is reduced by 1 Fr size compared to that of the sheath tube of the introducer sheath in the related art while maintaining the inner diameter of the sheath tube of the introducer sheath in the related art, by making the wall thickness of the sheath tube thin. For this reason, in comparison with the introducer sheath of the exemplary embodiment and the introducer sheath in the related art of which the sizes of the outer diameters of the sheath tubes are the same as each other, the inner diameter of the sheath tube of the introducer sheath of the exemplary embodiment is larger than that of the introducer sheath in the related art. Therefore, in the introducer sheath of the exemplary embodiment, when inserting a medical instrument into the introducer sheath, it is possible to introduce a medical instrument, the size of which is the same as that of the introducer sheath in the related art, into the introducer sheath with a smaller puncture hole than that of the introducer sheath in the related art. In addition, invasiveness into the vascular endothelium is low, and there is a low chance of blocking the blood vessel. That is, the introducer sheath of the exemplary embodiment has higher flexibility than the introducer sheath in the related art, and therefore, has low invasiveness into the vascular endothelium. Moreover, the outer diameter of the introducer sheath of the exemplary embodiment is smaller than that of the introducer sheath in the related art when the sizes of the inner diameters of the sheath tubes are the same as each other, and therefore, there is a low chance of blocking the blood vessel due to the introducer sheath. Accordingly, the time for which a patient stays in the hospital is shortened by reducing the outer diameter of the introducer sheath by 1 Fr size, and therefore, it is possible to reduce a physical stress on a patient and an economic burden on the hospital.

In the above-described exemplary embodiment of the disclosure, the introducer sheath (introducer sheath which has the same inner diameter as that of an introducer sheath in a conventional product through which it is possible to insert a medical instrument having an outer diameter of a 6 Fr size; and has the same outer diameter as that of an introducer sheath in a conventional product through which it is possible to insert a medical instrument having an outer diameter of a 5 Fr size) which has an inner diameter of a 6 Fr size and an outer diameter of a 5 Fr size in a conventional product; and the introducer sheath (introducer sheath which has the same inner diameter as that of an introducer sheath in a conventional product through which it is possible to insert a medical instrument having an outer diameter of a 7 Fr size; and has the same outer diameter as that of an introducer sheath in a conventional product through which it is possible to insert a medical instrument having an outer diameter of a 6 Fr size) which has an inner diameter of a 7 Fr size and an outer diameter of a 6 Fr size in a conventional product have been described, but these are merely examples. Introducer sheaths with other sizes, for example, an introducer sheath (introducer sheath which has the same inner diameter as that of an introducer sheath in a conventional product through which it is possible to insert a medical instrument having an outer diameter of a 5 Fr size; and has the same outer diameter as that of an introducer sheath in a conventional product through which it is possible to insert a medical instrument having an outer diameter of a 4 Fr size) which has an inner diameter of a 5 Fr size and an outer diameter of a 4 Fr size in a conventional product; or an introducer sheath (introducer sheath which has the same inner diameter as that of an introducer sheath in a conventional product through which it is possible to insert a medical instrument having an outer diameter of a 6 Fr size; and has the same outer diameter as that of an introducer sheath in a conventional product through which it is possible to insert a medical instrument having an outer diameter of a 5.5 Fr size) which has an inner diameter of a 6 Fr size and an outer diameter of a 5.5 Fr size in a conventional product which is classified as a so-called “half size” kind may also be used.

The detailed description above describes an introducer. The invention is not limited, however, to the precise embodiments and variations described. Various changes, modifications and equivalents can effected by one skilled in the art without departing from the spirit and scope of the invention as defined in the accompanying claims. It is expressly intended that all such changes, modifications and equivalents which fall within the scope of the claims are embraced by the claims.

What is claimed is:

1. An introducer sheath comprising:
a sheath tube which has a lumen structure continuing from a proximal portion to a distal portion; and
a sheath hub which is connected to the proximal portion of the sheath tube,
wherein a return angle of a kinked portion when the sheath tube is bent by 180° is 0° to 15°.

2. The introducer sheath according to claim 1,
wherein the return angle of the kinked portion when the sheath tube is bent by 180° is 2.5° to 12.5°.

3. The introducer sheath according to claim 1,
wherein a minor axis length/major axis length ratio of a cross section of a return shape of the kinked portion when the sheath tube is bent by 180° is 0.50 to 0.90.

4. An introducer sheath comprising:
a sheath tube which has a lumen structure continuing from a proximal portion to a distal portion; and
a sheath hub which is connected to the proximal portion of the sheath tube,
wherein a minor axis length/major axis length ratio of a cross section of a return shape of a kinked portion when the sheath tube is bent by 180° is 0.50 to 0.90.

5. The introducer sheath according to claim 4,
wherein a return angle of the kinked portion when the sheath tube is bent by 180° is 0° to 15°.

6. The introducer sheath according to claim 4,
wherein the minor axis length/major axis length ratio of the cross section of the return shape of the kinked portion when the sheath tube is bent by 180° is 0.55 to 0.85.

7. The introducer sheath according to claim 1,
wherein a wall thickness of the sheath tube is 0.050 mm to 0.140 mm.

8. The introducer sheath according to claim 7,
wherein the wall thickness of the sheath tube is 0.110 mm to 0.140 mm.

9. The introducer sheath according to claim 1,
wherein an inner diameter of the sheath tube is 1.9 mm to 2.5 mm.

10. The introducer sheath according to claim 1,
wherein the inner diameter of the sheath tube is 2.3 mm to 2.8 mm.

11. The introducer sheath according to claim 1,
wherein a kink generation angle of the sheath tube is 30° to 50°.

12. The introducer sheath according to claim 1,
wherein the introducer sheath is formed of ethylene tetrafluoroethylene polymer.

13. The introducer sheath according to claim 1,
wherein the introducer sheath has a hemostasis valve incorporated in the sheath hub.

14. An introducer assembly comprising the introducer sheath according to claim 1 and a dilator.

15. The introducer sheath according to claim 4, wherein a wall thickness of the sheath tube is 0.050 mm to 0.140 mm.

16. The introducer sheath according to claim 1, wherein the return angle of the kinked portion when the sheath tube is bent by 180 degrees is defined by an angle formed by an axis of the sheath tube on a distal side from the kinked portion and an axis of the sheath tube on a proximal side from the kinked portion 30 seconds to 1 minute after the sheath tube is released from one end and another end of the sheath tube being brought into contact with one another.

17. The introducer sheath according to claim 4,
wherein an inner diameter of the sheath tube is 1.9 mm to 2.5 mm.

18. The introducer sheath according to claim 4,
wherein the introducer sheath is formed of ethylene tetrafluoroethylene polymer.

19. An introducer assembly comprising the introducer sheath according to claim 4 and a dilator.

20. The introducer sheath according to claim 4,
wherein a kink generation angle of the sheath tube is 30° to 50°.
